SEQUENCE LISTING (5 sequences)

SEQ ID NO: 1 - protein, 617 aa, Artificial Sequence [sequence omitted]

SEQ ID NO: 2 - DNA, 1854 bp, Artificial Sequence
  Description of Artificial Sequence Synthetic construct
atggctcagcggatgacaacacagctgctgctccttctagtgtgggtggctgtagtaggggaggctcagacaaggattgcagaacaaaaactaataagcgaggaggacctgctgcagatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtggctgcagccatgagtggggctgggccctgggcagcctggcctttcctgcttagcctggccctaatgctgctgtggctgctcagctga

SEQ ID NO: 3 - DNA, 37 bp, Artificial Sequence
  Description of Artificial Sequence Synthetic primer
ccctgcagatggaagacgccaaaaacataaagaaagg

SEQ ID NO: 4 - DNA, 34 bp, Artificial Sequence
  Description of Artificial Sequence Synthetic primer
gctgcagccacggcgatctttccgcccttcttgg

SEQ ID NO: 5 - DNA, 1854 bp, Artificial Sequence; identical to SEQ ID NO: 2
  Description of Artificial Sequence Synthetic construct

(19) United States
(12) Patent Application Publication
(10) Pub. No.: US 2010/0015603 A1
(43) Pub. Date: Jan. 21, 2010

(54) Chimeric proteins for measuring atp concentrations in pericellular space and related screening method

(76) Inventors: Francesco Di Virgilio, Ferrara (IT); Simonetta Falzoni, Ferrara (IT); Patrizia Pellegatti, Ferrara (IT); Paolo Pinton, Ferrara (IT); Rosario Rizzuto, Ferrara (IT)

Correspondence Address: Young & Thompson, 745 South 23rd Street, Arlington, VA, 22202, US

(21) Appl. No.: 11/920,913

(22) PCT Filed: May 22, 2006
(86) PCT No.: PCT/IT2006/000383

§ 371 Date: Nov. 21, 2007

(30) Foreign Application Priority Data
May 23, 2005 (IT) RM2005A000252

(51) Int. Cl. C12Q 1/68; C07K 14/00; C12N 9/10; C12N 9/02; C12N 15/11; C12N 15/00; C12N 5/08; C12M 1/00

(52) U.S. Cl. 435/6; 530/350; 435/193; 435/189; 536/23.2; 435/320.1; 435/369; 435/367; 435/287.2

(57) ABSTRACT

The invention relates to luminescent chimeric proteins comprising a first N-terminal protein sequence, a second protein sequence and a third C-terminal protein sequence wherein:
(i) said first and said third protein sequence are a leader sequence and an anchor sequence belonging to at least a receptor localized on a plasma membrane site;
(ii) said second protein sequence encodes for the full-length or partial sequence of a photoprotein and is inserted in frame between said first and said third sequence (i); said chimeric protein being addressed to said plasma membrane site of the cell wherein it is expressed.

The present invention relates to chimeric proteins for the measurement of ATP concentrations into the pericellular space and related screening method.

ATP is now accepted as an ubiquitous extracellular messenger (Burnstock, 2004; Di Virgilio et al., 2004; Wang et al., 2004). Responses elicited by this nucleotide, depending on the concentration and the given P2R subtype expressed by the target cell, range from chemotaxis (Oshimi et al., 1999), to cell adhesion (Freyer et al., 1998), from cytokines release (Perregaux & Gabel, 1994), to neurotransmitter secretion (Illes & Norenberg, 1993), from activation of apoptosis (Zanovello et al., 1990), to stimulation of cell proliferation (Neary et al., 2003).

Further, several mediators (neurotransmifters, cytokines, hormones) work together to enhance or to reduce ATP release in the microenvironment directly close to cell plasma membrane wherein the factor carries out its paracrine or autocrine action, or like endogenous drug.

While it is generally agreed that many disease conditions (trauma, inflammation, ischemia) may lead to an increase in the extracellular ATP concentration as a consequence of mere cell lysis, the pathways that support non-lytic ATP release are less clear. Increasing attention is payed to those signals that alert the immune system during the early phases of tissue damage or pathogen invasion (Matzinger, 2002; La Sala et al., 2003; Skoberne et al., 2004). Intracellular nucleotides are considered likely candidates to this role for their ubiquitous distribution, high intracellular concentration, negligible extracellular levels under quiescent conditions, presence of specific receptors and ability to modulate dendritic cell differentiation. The additional feature described here, unveiling a non-lytic and self sustaining release mechanism, make ATP an even more appealing danger signal.

The very low extracellular levels under quiescent conditions, the quick increases caused by many different stimuli, the fast degradation in the extracellular space, and the presence of specific receptors make ATP an ideal extracellular messenger (Burnstock, 2004; Zimmermann, 2000). However, full appreciation of the role of ATP as an extracellular signal has been hampered by lack of proper probes for accurate measurement of the extracellular concentration. Most measurements are performed by using the standard luciferin/luciferase assay in off- or on-line settings. All off-line techniques measure ATP in the cell-free supernatants, and therefore after ATP has diffused and equilibrated into the incubation medium. On-line measurements give a more accurate estimate of the actual ATP levels reached close to the site of release, but still involve manipulations that may seriously affect the measurements. The prototypic ATP probe is firefly luciferase, a bioluminescent ATP-dependent enzyme that can detect ATP in the pico-millimolar range. Luciferase is mostly used to assay the ATP concentration in cell-free supernatants after cell or tissue stimulation. This procedure, albeit technically very simple, involves manipulations that can cause cell rupture or unwanted stimulation (sampling, centrifugation, recovery of the supernatants). Furthermore, these off-line measurements do not allow detection of rapidly changing localized ATP transients close to the surface of the plasma membrane. Previous observations have clearly shown that ATP levels measured in the proximity of the plasma membrane surface can be up to 10-20 fold higher than those measured in the bulk solution by the soluble luciferase assay (Beigi et al., 1999).

On-line measurements give a more accurate estimate of the actual ATP levels reached close to the site of release, but still involve manipulations that may seriously affect the measurements, for instance, by producing positive false and/or negative false.

For example, a protein A-luciferase chimera was engineered by Dubyak et al., to detect local ATP release at the membrane level (Beigi et al., 1999). Use of this probe involves coating of the cell surface with IgG to allow binding of the luciferase chimera. The cell-attached probe yielded an ATP release from thrombin-stimulated platelets 10-15 fold higher than those recorded by soluble luciferase under similar experimental conditions. This method requires the use of specific antibodies for target cells that may alter the physiological properties. Further it is known that antibodies fixed on plasma membrane incur redistribution and endocytosis. This make very difficult to ensure stable levels of membrane luciferase.

Recently, a biosensor system based on cells and fragments thereof expressing ATP sensible P2X channels placed near to a source of ATP (Hayashi et al., 2004), that allow to measure ATP release as a change in the recorded current. This methodology makes use of “patch clamp” electrophysiology technique, that is extremely complex (cell fragments or cells must be entire) and makes the method open to the presence of false positive or negative because of the existence of an intermediate step of channel opening. Thus, it results in a random and poorly repeatable measurement.

In the light of the above it is evident the need of a method for measuring of ATP levels in the pericellular space which is reliable, simple and repeatable and which allows more reliable measurements in comparison with the current methods.

The authors of the present invention have now created luminescent chimeric proteins able to localize on plasma membrane of the adopted cellular, to be used as probes for the measurement of actual ATP extracellular levels, highly selective as insensitive to all other ADP, UTP, UDP and GTP nucleotides.

Particularly, the method which employes these luminescent chimeric probes found by the authors has the following advantages: 1) chimeric probe is expressed as a plasma membrane protein, thus exposed to the actual environment wherein we aim to measure ATP; 2) chimeric probe can be engineered to be targeted to virtually any plasma membrane region, thus allowing the measurement of extracellular ATP at discrete plasma membrane sites; 3) genetic manipulation may allow to measure with this technique ATP levels in vivo in experimental models.

Such method for the measurement of ATP concentration in the periplasmalemmal space through the detection of a luminescent signal was carried out on cells expressing (es. HEK293 cells) the recombinant or native P2X₇ receptor (P2X₇R) transfected with the chimeric probes according to the invention to determine its function. Both cell types release large amounts of ATP (100 to 200 μM) in response to P2X₇R activation. This novel approach unveils a hitherto unsuspected non lytic pathway for the release of amounts of ATP into the extracellular milieu in the low micromolar to millimolar level, thus easily measurable.

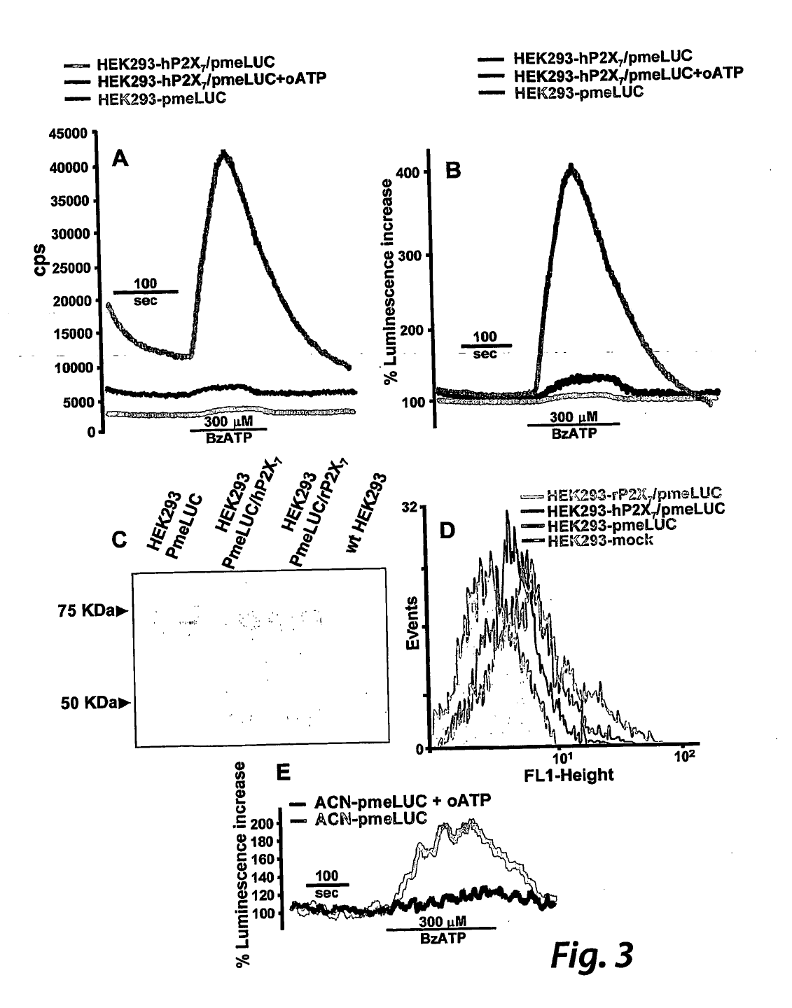

Indeed, the authors have shown that HEK293-pmeLUC transfectants did not appreciably release ATP in response to most stimuli applied. Expression of the P2X₇R on the contrary endows these cells with the ability to release large amounts of ATP in response to BzATP or ATP itself. The kinetic of BzATP-stimulated release is transient, reaching a peak within two min from the addition, and then rapidly declining to near basal level (see FIG. 3). This kinetic is surprising since it is well known that the P2X₇ is a non-desensitizing receptor, thus one would expect that so far the receptor stays open ATP should efflux. In fact opening of the P2X₇ pore allows to reach a periplasmalemmal ATP concentration in the hundred micromolar range, sufficient to activate even the low affinity P2X₇R. This observation supports the hypothesis that, once an initial event triggers ATP release to a level sufficient to activate P2X₇, neighbour cells expressing this receptor (mainly inflammatory cells) may function as amplification devices by sustaining a process of ATP-induced ATP release.

Hence these cellular systems transfected with the probes according to the invention may be used in screening method for the evaluation of drugs or molecules of interest that may cause a reduction or an increase of ATP levels in the periplasmalemmal space.

Furthermore these systems may be used as biosensors to detect the presence of toxic substances and/or environmental contaminants.

Thus, it is an object of the invention luminescent chimeric proteins comprising a first N-terminal protein sequence, a second protein sequence and a third C-terminal protein sequence wherein:

(i) said first and said third protein sequence are a leader sequence and an anchor sequence belonging to at least a receptor localized on a plasma membrane site;
(ii) said second protein sequence encodes for a photoprotein and is inserted in frame between said first and said third sequence (i); said chimeric protein being addressed to said plasma membrane site of the cell wherein it is expressed.

Preferably, the receptor localized on the plasma membrane site is selected from the group that consists in ionic-channel receptors, connexins, G protein coupled receptors, tyrosine-kinase activity receptors. More preferably, said photoprotein is selected from the group that consists in luciferase, aequorin, obelin.

According to a preferred embodiment of the present invention said first and said third protein sequence (i) are the leader sequence and the GPI anchor sequence of the folate receptor and said photoprotein (ii) is luciferase, preferably fire-fly luciferase. Most preferably the aminoacid sequence of the protein according to the invention, namely pMeLuc, is the following:

(SEQ ID No: 1)

MAQRMTTQLLLLLVWVAVVGEAQTRIAEQKLISEEDLLQMEDAKNIKKG
PAPFYPLEDGTAGEQLHKAMKRYALVPGTIAFTDAHIEVDITYAEYFEM
SVRLAEAMKRYGLNTNHRIVVCSENSLQFFMPVLGALFIGVAVAPANDI
YNERELLNSMGISQPTVVFVSKKGLQKILNVQKKLPIIQKIIIMDSKTD
YQGFQSMYTFVTSHLPPGFNEYDFVPESFDRDKTIALIMNSSGSTGLPK
GVALPHRTACVRFSHARDPIFGNQIIPDTAILSVVPFHHGFGMFTTLGY
LICGFRVVLMYRFEEELFLRSLQDYKIQSALLVPTLFSFFAKSTLIDKY
DLSNLHEIASGGAPLSKEVGEAVAKRFHLPGIRQGYGLTETTSAILITP
EGDDKPGAVGKVVPFFEAKVVDLDTGKTLGVNQRGELCVRGPMIMSGYV
NNPEATNALIDKDGWLHSGDIAYWDEDEHFFIVDRLKSLIKYKGYQVAP
AELESILLQHPNIFDAGVAGLPDDDAGELPAAVVVLEHGKTMTEKEIVD
YVASQVTTAKKLRGGVVFVDEVPKGLTGKLDARKIREILIKAKKGGKIA
VAAAMSGAGPWAAWPFLLSLALMLLWLLS.

It is a further object of the present invention a nucleotidic sequence encoding for one of the luminescent chimeric proteins luminescenti as above defined. Particularly, among the nucleotidic sequences encoding the above mentioned pmeLuc aminoacid sequence is preferably used the following nucleotidic sequence: ATGGCTCAGCGGATGACAACACAGCTGCTGCTCCTTCTAGTGTGGGT GGCTGTAGTAGGGGAGGCTCAGACAAGGATTGCAGAACAAAAACTAA TAAGCGAGGAGGACCTGCTGCAGATGGAAGACGCCAAAACATAAA GAAAGGCCCGGCGCCATTCTATCCGCTGGAAMGATGGAACCGCTGGA GAGCAACTGCATAAGGCTATGAAGAGATACGCCCTGGTTCCTGGAAC AATTGCTTTTACAGATGCACATATCGAGGTGGACATCACTTACGCTGA GTACTTCGAAATGTCCGTTCGGTTGGCAGAAGCTATGAAACGATATG GGCTGAATACAAATCACAGAATCGTCGTATGCAGTGAAAACTCTCTTC AATTCTTTATGCCGGTGTTGGGCGCGTTATTTATCGGAGTTGCAGTTG CGCCCGCGAACGACATTTATAATGAACGTGAATTGCTCAACAGTATG GGCATTTCGCAGCCTACCGTGGTGTTCGTTTCCAAAAAGGGGTTGCA AAAAATTTTGAACGTGCAAAAAAAGCTCCCAATCATCCAAAAAATTATT ATCATGGATTCTAAAACGGATTACCAGGGATTTCAGTCGATGTACACG TTCGTCACATCTCATCTACCTCCCGGTTTTAATGAATACGATTTTGTG CCAGAGTCCTTCGATAGGGACAAGACAATTGCACTGATCATGAACTC CTCTGGATCTACTGGTCTGCCTAAAGGTGTCGCTCTGCCTCATAGAA CTGCCTGCGTGAGATTCTCGCATGCCAGAGATCCTATTTTTGGCAAT CAAATCATTCCGGATACTGCGATTTTAAGTGTTGTTCCATTCCATCAC GGTTTTGGAATGTTTACTACACTCGGATATTTGATATGTGGATTTCGA GTCGTCTTAATGTATAGATTTGAAGAAGAGCTGTTTCTGAGGAGCCTT CAGGATTACAAGATTCAAAGTGCGCTGCTGGTGCCAACCCTATTCTC CTTCTTCGCCAAAAGCACTCTGATTGACAAATACGATTTATCTAATTTA CACGAAATTGCTTCTGGTGGCGCTCCCCTCTCTAAGGAAGTCGGGGA AGCGGTTGCCAAGAGGTTCCATCTGCCAGGTATCAGGCAAGGATATG GGCTCACTGAGACTACATCAGCTATTCTGATTACACCCGAGGGGGAT GATAAACCGGGCGCGGTCGGTAAAGTTGTTCCATTTTTTGAAGCGAA GGTTGTGGATCTGGATACCGGGAAAACGCTGGGCGTTAATCAAAGAG GCGAACTGTGTGTGAGAGGTCCTATGATTATGTCCGGTTATGTAAAC AATCCGGAAGCGACCAACGCCTTGATTGACAAGGATGGATGGCTACA TTCTGGAGACATAGCTTACTGGGACGAAGACGAACACTTCTTCATCG TTGACCGCCTGAAGTCTCTGATTAAGTACAAAGGCTATCAGGTGGCT CCCGCTGAATTGGAATCCATCTTGCTCCAACACCCCAACATCTTCGA CGCAGGTGTCGCAGGTCTTCCCGACGATGACGCCGGTGAACTTCCC GCCGCCGTTGTTGTTTTGGAGCACGGAAAGACGATGACGGAAAAAGA GATCGTGGATTACGTCGCCAGTCAAGTAACAACCGCGAAAAAGTTGC GCGGAGGAGTTGTGTTTGTGGACGAAGTACCGAAAGGTCTTACCGG AAAACTCGACGCAAGAAAAATCAGAGAGATCCTCATAAAGGCCAAGA AGGGCGGAAAGATCGCCGTGGCTGCAGCCATGAGTGGGGCTGGGC CCTGGGCAGCCTGGCCTTTCCTGCTTAGCCTGGCCCTAATGCTGCTG TGGCTGCTCAGCTGA (SEQ ID No:2). This nucleotidic sequence is preferably used into the VR012 vector.

It is an object of the present invention an expression vector comprising the nucleotidic sequence as above defined, preferably pcDNA3 or VR1012.

The present invention provides a primary cell culture transfected with the above mentioned expression vector or with the nucleotidic sequence according to the invention, such as fibroblasts isolated from skin or microglial cells isolated from cerebral tissue of newborn mice.

It is further object of the present invention a cell line transfected with the above defined expression vector or with the nucleotidic sequence according to the invention. More preferably, said cell line is characterized by the native or recombinant expression of a receptor of interest preferably selected between CD14, P2Y (several subtypes) and P2X (several subtypes), preferably P2X₇. Preferably, the cell line is selected between HEK 293, HeLa, ACN, N9, N13, PC12, J774, A549. These two latter cell lines constitutively expressing both P2X₇, and CD14 receptor represent a particularly advantageous cellular system to be employed. Particularly, the macrophage cell line may be considered particularly advantageous in the systems for the analysis of substances toxicity due to macrophage ability to phagocytize toxic substances. Parallely the pulmonary epithelial cells may be considered particularly advantageous in the systems for the analysis of environmental contaminants because of their specific location at level of respiratory airways.

The present invention concerns the use of luminescent chimeric proteins according to the invention, as probes for the measurement of extracellular ATP levels into in vitro or in vivo systems.

It is a further object of the invention the use of cell lines as above defined for the measurement of extracellular ATP levels in in vitro systems or alternatively as experimental model in screening method and/or analysis method of compounds of interest able to modulate extracellular ATP levels.

Finally, it is an object of the present invention a screening method of compounds of interest (e.g. anti-inflammatory drugs, immunomodulators, vasodilators) able to modulate extracellular ATP levels comprising the following steps:

a) contacting the cell line as above defined wherein the photoprotein is luciferase with said compound of interest in the presence of O₂, Mg²⁺ and of the luciferin substrate;
b) detecting cps or luminescence percent over basal value or the value of a preceding stimulation with an agonist as a control and determining the activity as an agonist or antagonist in relation to cps increase or reduction over basal value or the value of a preceding stimulation with an agonist as a control, respectively.

According to a preferred embodiment the present invention relates to a method for the analysis of the presence of toxic substances and/or environmental contaminants able to induce the increase of extracellular ATP levels as an early index of cell suffering in a measure proportional to the toxicity of the substance and to the exposure time, comprising the following steps:

a) contacting a cell line sensible to the toxic substance(s) to be assayed as above defined when the photoprotein is luciferase with said compound of interest in the presence of O₂, Mg²⁺ and of the luciferin substrate;
b) detecting the presence or the absence of a toxic substance in relation to the cps or luminescence percent increase or reduction over the basal value.

Preferably, when the analysis method is for the detection of LPS and/or ozone the cell line of step a) is selected between pulmonary epithelial cells, and macrophage cells of mammalian origin, preferably of human origin. Preferably, said human pulmonary epithelial cells are A459 cells. Preferably, said human macrophage cells are J774 cells.

According to a preferred embodiment of the analysis method of the invention said toxic substance to be assayed is LPS and/or ozone. In a particularly preferred embodiment of the invention if ozone and/or LPS are assayed the above mentioned A549 and/or J774 cell lines are employed.

The present invention refers to a biosensor comprising a cell line a cell line characterized in that it is transfected with the above defined expression vector or with the nucleotidic sequence according to the invention. Said line may be further characterized by the native or recombinant expression of a receptor (e.g. P2X₇). Preferably, said cell line is selected from the group that consists in HEK 293, HeLa, ACN, N9, N13, PC12, J774, A549.

The invention refers to the use of the above mentioned biosensor for the measurement of extracellular ATP levels in in vitro systems, for example for the analysis of environmental toxicity or for the screening of compounds of interest (e.g. anti-inflammatory drugs, immunomodulators, vasodilators) able to alter extracellular ATP levels.

According to an alternative embodiment of biosensor, the present invention further concerns a biosensor comprising at least one of the luminescent chimeric proteins contemplated by the present invention. Finally, the invention concerns the use of the above mentioned biosensor for the measurement of extracellular ATP levels in in vitro systems for example for the analysis of environmental toxicity or for the screening of compounds of interest (e.g. anti-inflammatory drugs, immunomodulators, vasodilators) able to alter extracellular ATP levels.

The present invention will be now described for illustrative but non-limiting purposes, according to its preferred embodiments, with particular reference to the figures of the enclosed drawings, in which:

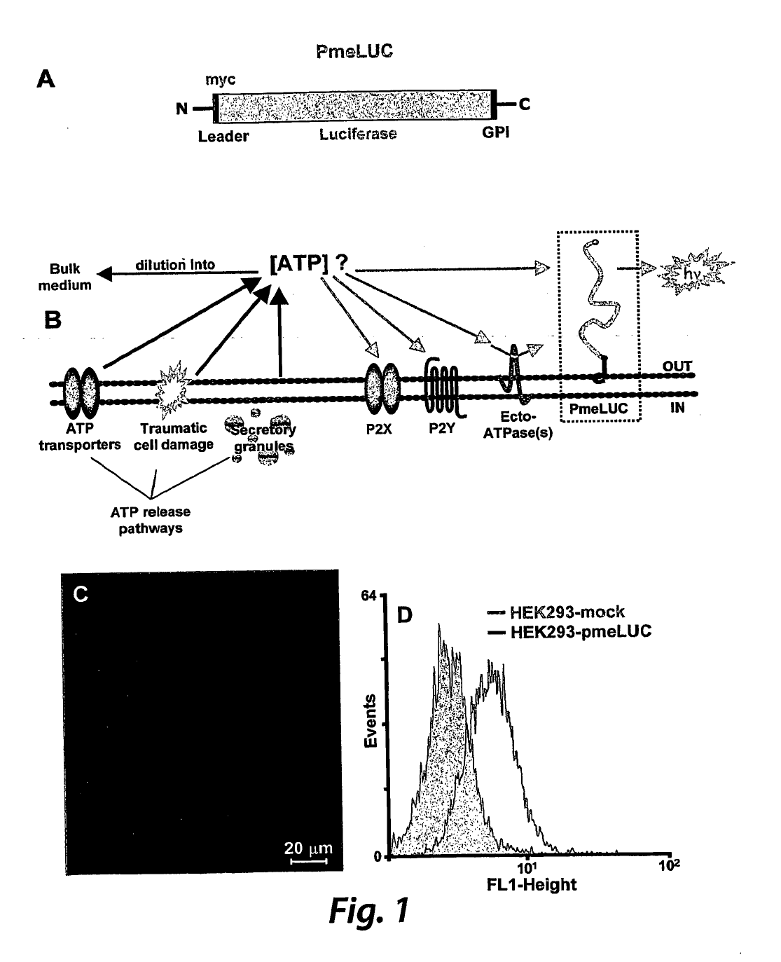

FIG. 1 shows the structure and localization of pmeLUC construct; panel A shows the structure comprising the full length coding sequence of luciferase inserted in frame between the N-terminal, leader sequence (26 aa) and the C-terminal GPI anchor sequence (28 aa) of the folate receptor, panel B shows a schematic rendering of the plasma membrane localization of pmeLUC; panels C and D reproduce immunofluorescence and FACS analysis of HEK293 cells transfected with pmeLUC (HEK293-pmeLUC) or with the empty vector (HEK293-mock), respectively;

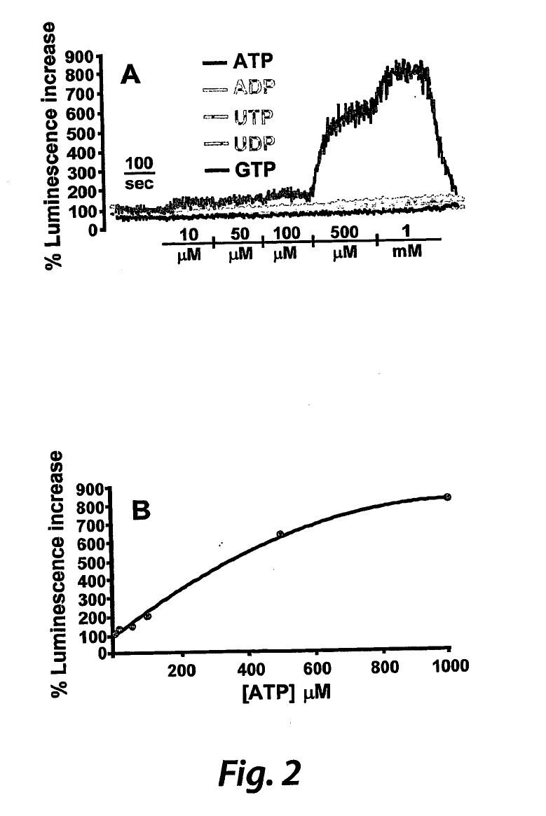

FIG. 2 shows the response of HEK293-pmeLUC cells to extracellular nucleotides in panel A, and ATP calibration curve in panel B; HEK293-pmeLUC cell monolayers were placed in the luminometer chamber, and were then perfused with solutions containing increasing concentrations of nucleotides; basal cps before addition of the nucleotides ranged between 2500 and 3200; luminescence increase is shown as percent increase over basal; in panel B luminescence increase is correlated to the ATP concentration to build a calibration curve;

FIG. 3 shows ATP release through the P2X₇ receptor; panels A and B show HEK293 cells co-transfected with hP2X₇ and pmeLUC (HEK293-hP2X₇/pmeLUC) placed in the luminometer chamber and perfused with a BzATP-containing solution, with or without prior treatment with 300 μM oATP for 2 hours; HEK293-pmeLUC cells are used as a control; luminescence increase was expressed as cps in panel A and luminescence percent increase over basal in panel B; in panel C total pmeLUC protein expressed in HEK293-pmeLUC, HEK293-hP2X₇/pmeLUC and HEK293-rP2X₇/pmeLUC cells is determined by western blotting wherein wtHEK293 cells are shown as a control; in panel (D) plasma membrane-expressed pmeLUC is measured by FACS analysis in these three cell populations; panel E shows ATP release from human neuroblastoma ACN cells incubated in the absence or presence of 300 μM oATP;

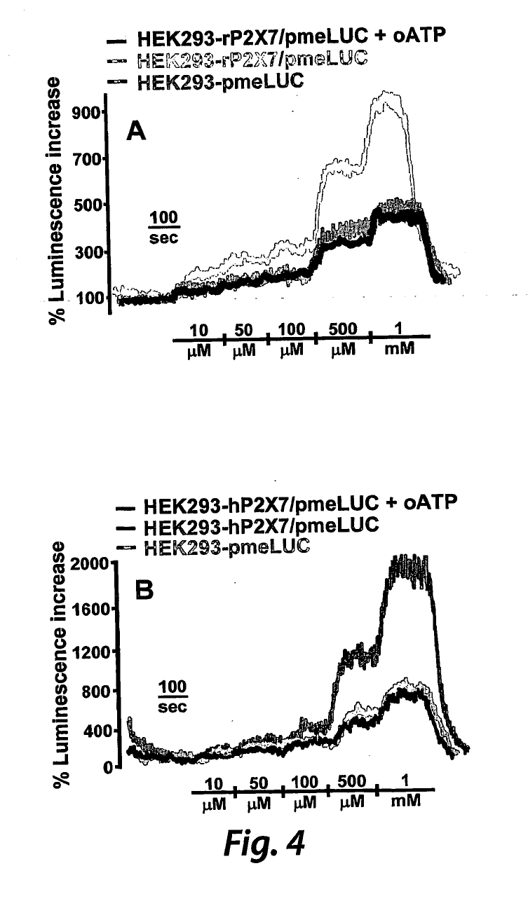

FIG. 4 shows extracellular ATP release from P2X₇-expressing cells. HEK293 cells transfected with the rat (panel A) or human (panel B) P2X₇R and control cells were placed in the luminometer chamber and perfused with increasing ATP concentrations;

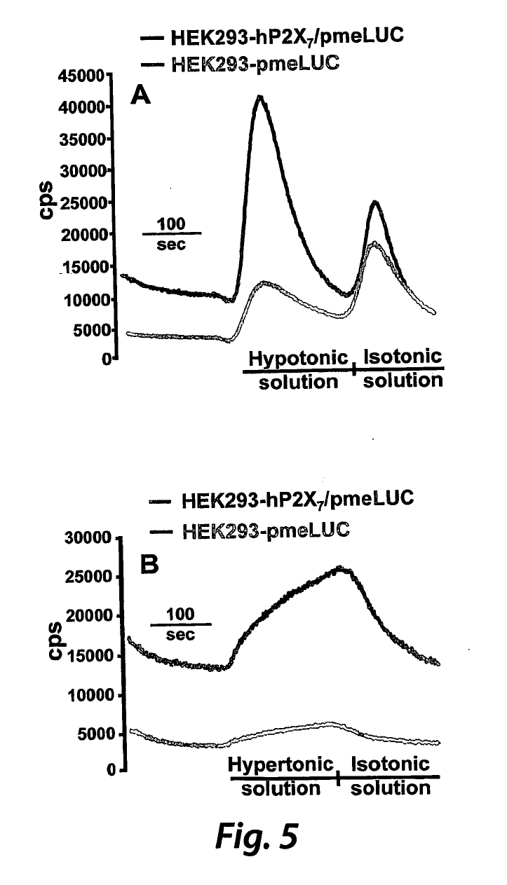

FIG. 5 shows the increase of ATP release triggered by membrane stretching due to the expression of the P2X₇R; in panel A HEK293-hP2X₇/pmeLUC and HEK293-pmeLUC cells were placed in the luminometer chamber and perfused with isotonic buffer followed by a hypotonic solution, followed again by isotonic buffer; in panel B HEK293-hP2X₇/pmeLUC and HEK293-pmeLUC cells were perfused with isotonic buffer followed by a hypertonic solution;

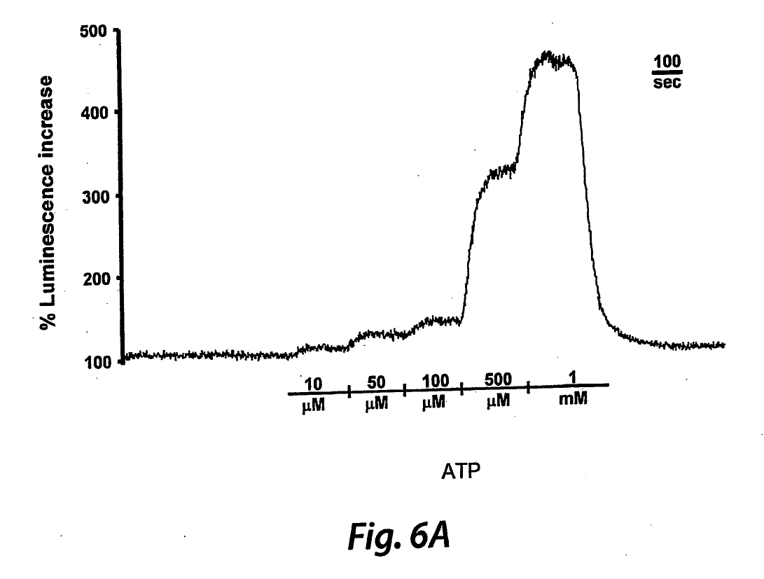

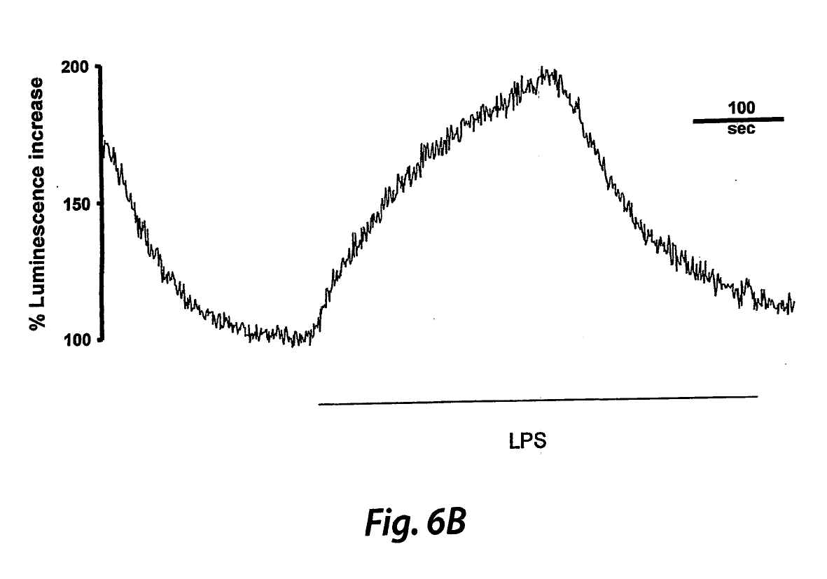

FIGS. 6 A and 6 B show two diagram showing the measurement of ATP release from J774 cells expressing pmeLUC following exposure to different substances (panel A: treatment with different ATP concentrations ranging from 5 μM to 1 mM; panel B: treatment with LPS);

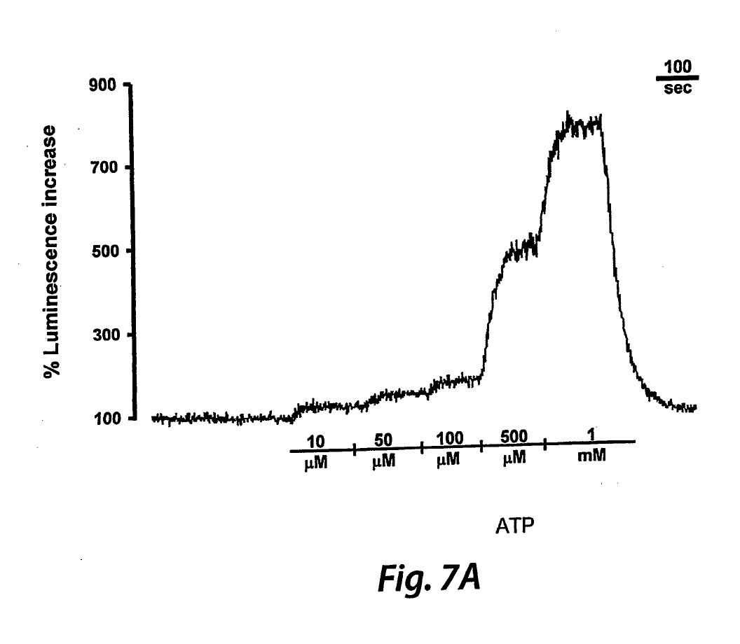

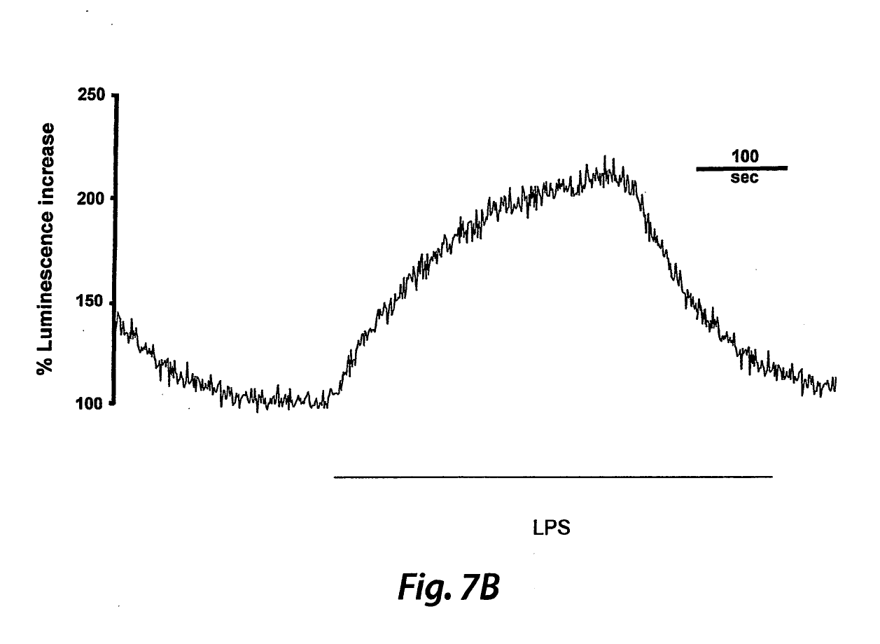

FIGS. 7 A and 7 B show two diagram showing the measurement of ATP release from A459 cells expressing pmeLUC following exposure to different substances (panel A: treatment with different ATP concentrations ranging from 5 μM to 1 mM; panel B: treatment with LPS 1 μg/ml).

### EXAMPLE 1

### Preparation of the Luminescent Chimneric Probe pMeLuc for ATP Measurements and HEK293-P2X₇ Cells Transfected with said Probe

### Materials and Methods

Benzoyl ATP (BzATP), oxidized ATP (oATP), DMEM, DMEM-F12, MEM non-essential amino acid solution 100× were from Sigma-Aldrich (St. Louis, Mo., USA). ATP, ADP, UTP, UDP and GTP were purchased from Boehringer-Roche Diagnostics (Mannheim, Germany). Luciferin used for ATP measurements with pmeLUC was from DUCHEFA Biochemie (Amsterdam, The Netherlands). Luciferin-luciferase solutions for ATP measurements with the Firezyme luminometer were from Promega (Madison, Wis., USA). Dithiothreitol (DTT) was purchased from Merck (Damstadt, Germany). All experiments were performed in a saline solution containing: 135 mM NaCl, 5 mM KCl, 0.5 mM KH₂PO₄, 1 mM MgSO4, 1 mM CaCl₂, 5.5 mM glucose and 20 mM Hepes, pH 7.4 at 37° C.

### PmeLUC Engineering

Plasma membrane luciferase pmeLUC was obtained as follows: luciferase cDNA was amplified from a modified pGL3 plasmid (kind gift of Dr Guy Rutter, University of Bristol, UK) using the following primers: 5′-CCC TGC AGA TGG MG ACG CCA AAA ACA TAA AGA MG G 5′-CCC TGC AGA TGG MG ACG CCA AAA ACA TM AGA MG G-3′(SEQ ID No:3) (corresponding to the sequence encoding amino acids 1-9 of luciferase; PstI site underlined) and 5′-GCT GCA GCC ACG GCG ATC TIT CCG CCC TTC TTG G-3′ (SEQ ID No:4) (including amino acids 542-549 of luciferase cDNA without the stop codon; PstI site underlined).

The PCR product was transferred to pBSK⁺ vector (Stratagene), digested with the enzyme PstI and inserted in the right frame between a PstI fragment encoding the complete N-terminal leader sequence of the human folate receptor (26 aa) fused with myc tag (10 aa) and a PstI fragment of the GPI anchor protein (28 aa), to generate the construct shown in FIG. 1A. Particularly, the nucleotidic sequence used was the following:

(SEQ ID No: 5)

ATGGCTCAGCGGATGACAACACAGCTGCTGCTCCTTCTAGTGTGGG
TGGCTGTAGTAGGGGAGGCTCAGACAAGGATTGCAGAACAAAAACT
AATAAGCGAGGAGGACCTGCTGCAGATGGAAGACGCCAAAAACATA
AAGAAAGGCCCGGCGCCATTCTATCCGCTGGAAGATGGAACCGCTG
GAGAGCAACTGCATAAGGCTATGAAGAGATACGCCCTGGTTCCTGG
AACAATTGCTTTTACAGATGCACATATCGAGGTGGACATCACTTACG
CTGAGTACTTCGAAATGTCCGTTCGGTTGGCAGAAGCTATGAAACGA
TATGGGCTGAATACAAATCACAGAATCGTCGTATGCAGTGAAAACTC
TCTTCAATTCTTTATGCCGGTGTTGGGCGCGTTATTTATCGGAGTTG
CAGTTGCGCCCGCGAACGACATTTATAATGAACGTGAATTGCTCAAC
AGTATGGGCATTTCGCAGCCTACCGTGGTGTTCGTTTCCAAAAAGG
GGTTGCAAAAAATTTTGAACGTGCAAAAAAAGCTCCCAATCATCCAA
AAAATTATTATCATGGATTCTAAAACGGATTACCAGGGATTTCAGTCG
ATGTACACGTTCGTCACATCTCATCTACCTCCCGGTTTTAATGAATAC
GATTTTGTGCCAGAGTCCTTCGATAGGGACAAGACAATTGCACTGAT
CATGAACTCCTCTGGATCTACTGGTCTGCCTAAAGGTGTCGCTCTGC
CTCATAGAACTGCCTGCGTGAGATTCTCGCATGCCAGAGATCCTATT
TTTGGCAATCAAATCATTCCGGATACTGCGATTTTAAGTGTTGTTCCA
TTCCATCACGGTTTTGGAATGTTTACTACACTCGGATATTTGATATGT
GGATTTCGAGTCGTCTTAATGTATAGATTTGAAGAAGAGCTGTTTCT
GAGGAGCCTTCAGGATTACAAGATTCAAAGTGCGCTGCTGGTGCCA
ACCCTATTCTCCTTCTTCGCCAAAAGCACTCTGATTGACAAATACGAT
TTATCTAATTTACACGAAATTGCTTCTGGTGGCGCTCCCCTCTCTAA
GGAAGTCGGGGAAGCGGTTGCCAAGAGGTTCCATCTGCCAGGTATC
AGGCAAGGATATGGGCTCACTGAGACTACATCAGCTATTCTGATTAC
ACCCGAGGGGGATGATAAACCGGGCGCGGTCGGTAAAGTTGTTCC
ATTTTTTGAAGCGAAGGTTGTGGATCTGGATACCGGGAAAACGCTG
GGCGTTAATCAAAGAGGCGAACTGTGTGTGAGAGGTCCTATGATTAT
GTCCGGTTATGTAAACAATCCGGAAGCGACCAACGCCTTGATTGAC
AAGGATGGATGGCTACATTCTGGAGACATAGCTTACTGGGACGAAG
ACGAACACTTCTTCATCGTTGACCGCCTGAAGTCTCTGATTAAGTAC
AAAGGCTATCAGGTGGCTCCCGCTGAATTGGAATCCATCTTGCTCCA
ACACCCCAACATCTTCGACGCAGGTGTCGCAGGTCTTCCCGACGAT
GACGCCGGTGAACTTCCCGCCGCCGTTGTTGTTTTGGAGCACGGAA
AGACGATGACGGAAAAAGAGATCGTGGATTACGTCGCCAGTCAAGT
AACAACCGCGAAAAAGTTGCGCGGAGGAGTTGTGTTTGTGGACGAA
GTACCGAAAGGTCTTACCGGAAAACTCGACGCAAGAAAAATCAGAG
AGATCCTCATAAAGGCCAAGAAGGGCGGAAAGATCGCCGTGGCTGC
AGCCATGAGTGGGGCTGGGCCCTGGGCAGCCTGGCCTTTCCTGCT
TAGCCTGGCCCTAATGCTGCTGTGGCTGCTCAGCTGA.

Legend: leader sequence is shown in italics; myc epitope is indicated in bold; fire-fly luciferase sequence is indicated in capitals; GPI sequence of folate receptor is underlined.

The whole final construct was excised by a NotI/XhoI or XbaI digestion, and cloned into the expression vectors pcDNA3 or VR1012, respectively. The clone was checked by sequence analysis carried out on service at the Bio Molecular Research sequencing core of the CRIBI-University of Padova.

### Cell Transfection

HEK293 cells were cultured in DMEM-F12 (Sigma-Aldrich). Media were supplemented with 10% heat-inactivated FBS, 100 U/ml penicillin and 100 μg/ml streptomycin (Invitrogen corporation, San Giuliano Milanese, Italy). ACN neuroblastoma cells were cultured in DMEM supplemented with MEM non-essential amino acid solution 100× (Sigma-Aldrich). HEK293 cells were transfected with the calcium phosphate method. Cells transiently expressing the pmeLUC construct were assayed 36 hours after transfection. Clones stably expressing pmeLUC or the P2X₇R were generated by culture of the transfected cells in the presence of G418 (0.8 mg/ml, added 48 hours after transfection) for 3 weeks. Stable P2X₇- or pmeLUC-expressing clones were kept in the continuous presence of 0.2 mg/ml of G418 sulphate (Geneticin) (Calbiochem, la Jolla, Calif.). ACN cells were transfected with pmeLUC by lipofectamine (Invitrogen) and tested 24 hours after transfection. Briefly, cells were incubated in 250 μl serum-free transfection medium (OPTIMEM) in the presence of lipofectamine-plus-DNA (0.4 μg per well). After 3 hours incubation 1 ml of DMEM plus 10% FBS supplemented with MEM non-essential amino acid solution 100× (Sigma-Aldrich) was added. Cells were assayed 24 hours after transfection. In order to allow a high level of plasma membrane expression of the transfected constructs, cells were incubated overnight in the presence of 1 mM DTT (Mezghrani et al, 2001). Furthermore, they were also kept at room temperature (21° C.) for two hours prior to transfer into the thermostated luminometer chamber. These treatments that did not perturb luciferase activity or P2X₇ function maximized pmeLUC surface expression by improving transport to the plasma membrane and slowing down recycling.

### Immunofluorescence

HEK293 cells, seeded onto 24 mm coverslips, were fixed with 4% formaldehyde in PBS solution for 30 minutes, permeabilized with 0.2% Triton X-100 for 5 minutes at room temperature, rinsed three times with PBS, and incubated for 30 minutes with 0.2% gelatine in PBS to block non specific binding-sites. Immunostaining was carried out for 1 hour at 37° C. with a commercial monoclonal antibody against the c-myc epitope tag (Santa Cruz Biotechnology Inc., CA, USA) at a 1:100 dilution in 0.2% gelatine in PBS. Immunodetection was carried out using Texas-Red conjugated goat anti-mouse IgG (Santa Cruz) used at 1:50 dilution in 0.2% gelatine in PBS. After immunostaining, cells were imaged with a Zeiss LSM 510 Confocal Laser Scanning Microscope.

### ATP Measurement

ATP was measured in the custom-made luminometer described by Rizzuto and co-workers (Brini et al., 1999; Jouaville et al., 1999). For experiments, cells were plated onto 13 mm coverslips and were placed in a 37° C. thermostatted chamber (diameter 15 mm, height 2 mm) and perfused with a saline solution supplemented with luciferin at a concentration of 5 μM. The chamber was held in a photomultiplier kept in a dark refrigerated (4° C.) box. Light emission was detected by a Thorn EMI photon counting board installed in an IBM-compatible computer. The board allowed storing of the data in the computer memory for further analysis. During the experiments the thermostatted chamber was continuously perfused with buffer by means of a Gilson peristaltic pump. Alternatively, ATP was measured in the supernatants using soluble luciferase in a Firezyme luminometer as previously described (Baricordi et al., 1999; Solini et al, 2004; Zanovello et al., 1990).

### FACS Analysis

Non-permeabilized HEK293 cells stably transfected with pmeLUC or with the empty vector were labeled with the murine monoclonal antibody (Santa Cruz) directed against the pmeLUC c-myc tag at a 1:100 dilution in PBS for 1 hour at 4° C. At the end of this incubation, cells were incubated with FITC-conjugated anti-mouse antibody at a 1:50 dilution in PBS for 1 hour at 4° C. Fluorescence emission was analysed with an argon laser cytofluorometer FACS Scan Vantage (Beckton Dickinson, Franklin Lakes, N.J., USA).

### Results

The structure of the novel ATP probe (pmeLUC) is shown in FIG. 1A. This chimeric protein, thanks to the folate receptor leader sequence, is targeted to the plasma membrane and detects ATP in the extracellular space close to the cell surface (FIG. 1B). The aminoacid sequence of pmeLuc is preferably:

(SEQ ID No: 1)

MAQRMTTQLLLLLVWVAVVGEAQTRIAEQKLISEEDLLQMEDAKNIKKG
PAPFYPLEDGTAGEQLHKAMKRYALVPGTIAFTDAHIEVDITYAEYFEM
SVRLAEAMKRYGLNTNHRIVVCSENSLQFFMPVLGALFIGVAVAPANDI
YNERELLNSMGISQPTVVFVSKKGLQKILNVQKKLPIIQKIIIMDSKTD
YQGFQSMYTFVTSHLPPGFNEYDFVPESFDRDKTIALIMNSSGSTGLPK
GVALPHRTACVRFSHARDPIFGNQIIPDTAILSVVPFHHGFGMFTTLGY
LICGFRVVLMYRFEEELFLRSLQDYKIQSALLVPTLFSFFAKSTLIDKY
DLSNLHEIASGGAPLSKEVGEAVAKRFHLPGIRQGYGLTETTSAILITP
EGDDKPGAVGKVVPFFEAKVVDLDTGKTLGVNQRGELCVRGPMIMSGYV
NNPEATNALIDKDGWLHSGDIAYWDEDEHFFIVDRLKSLIKYKGYQVAP
AELESILLQHPNIFDAGVAGLPDDDAGELPAAVVVLEHGKTMTEKEIVD
YVASQVTTAKKLRGGVVFVDEVPKGLTGKLDARKIREILIKAKKGGKIA
VAAAMSGAGPWAAWPFLLSLALMLLWLLS.

Immunofluoresence and FACS analysis of cells transfected with this construct confirm that pmeLUC is expressed on the plasma membrane (FIGS. 1C and D). Cells expressing pmeLUC have a basal level of luminescence emission that depends on the amount of expressed luciferase, however in the stable HEK293-pmeLUC clones generated and used by the authors of the invention in the experiments, basal luminescence emission was comprised within a fairly narrow cps (counts per second) range (i.e. from a lower emission of about 2500 cps, to a higher of about 3200 cps).

To minimize variations due to minor changes in pmeLUC expression luminescence can be expressed as percent increase over basal, as shown in FIG. 2A, where HEK293-pmeLUC cells are challenged with different nucleotides in order to test affinity and selectivity of the probe. Affinity of pmeLUC for ATP is rather low, with a threshold of about 10 μM, however, subsequent ATP additions elicit further increases in light emission that allow building a calibration curve (FIG. 2B). Importantly, pmeLUC is insensitive to all other nucleotides tested (ADP, UTP, UDP and GTP) (FIG. 2A). To check for ability of pmeLUC to monitor ATP release triggered by receptor-directed stimuli, the HEK293-pmeLUC cells were challenged with various agonists of G protein-coupled receptors (e.g. carbachol, histamine, bradykinin), obtaining negligible ATP release (not shown).

Early experiments carried out not only by the authors of the present invention had shown that supernatants from cells expressing the P2X₇ receptor contained a high level of ATP, which was not due to an accelerated rate of cell lysis (Baricordi et al., 1999; Adinolfi et al., 2003; Solini et al., 2004). This suggested that the P2X₇R might be one of the pathways mediating ATP translocation across the plasma membrane. To test such hypothesis several HEK293 clones were generated stably transfected with the human or rat P2X₇R (HEK293-hP2X₇ or HEK293-rP2X₇, respectively). These clones were then transfected with pmeLUC (HEK293-P2X₇/pmeLUC). As shown in FIG. 3A, the HEK293-hP2X₇/pmeLUC cells exhibit a several fold higher level of basal fluorescence compared to HEK293-pmeLUC (12250±1500 versus 2300±850 cps, respectively, n=8). Addition of BZATP, a potent P2X₇ agonist, triggers a large luminescence increase in the HEK293-hP2X₇/pmeLUC or HEK293-rP2X₇/pmeLUC (only HEK293-hP2X₇/pmeLUC shown), but not in the HEK293-pmeLUC cells. The luminescence increase triggered by BZATP is fully blocked by pre-treatment with oxidized ATP (oATP), a powerful and irreversible blocker of the P2X₇R (Murgia et al., 1993).

To rule out a possible inhibitory effect of oATP on the luciferase itself, a calibration curve was performed in the presence and absence of this inhibitor, showing that the ATP-dependent luminescence increase is not affected (not shown but see also FIG. 4). Calibration of BzATP triggered luminescence increase yields a peak ATP concentration of about 250 μM.

Differences in basal levels of luminescence emission are neutralized by expressing luminescence as percentage increase over basal (FIG. 3B), however, the higher basal luminescence of HEK293-hP2X₇/pmeLUC might reflect a real increased level of pericellular ATP compared to HEK293-pmeLUC, rather than a higher expression of pmeLUC. In this case, reporting luminescence as percent increase over basal would mask such a difference in the extracellular ATP concentration. To clarify this issue, total pmeLUC protein was measured by western blotting (FIG. 3C). Blots show that, although there is some variability in protein expression in the different stable transfectants, HEK293-hP2X₇/pmeLUC or HEK293-rP2X₇/pmeLUC have a lower content of luciferase compared to HEKpmeLUC, and this cannot account for the higher basal luminescence emission in the P2X₇-transfected clones. To measure quantitatively surface pmeLUC expression, different clones were analyzed by FACS. As shown in FIG. 3D, pmeLUC expression profiles of HEK293-hP2X₇/pmeLUC, HEK293-rP2X₇/pmeLUC and HEK293-pmeLUC closely overlaps. Therefore, in keeping with previous findings (Baricordi et al., 1999; Solini et al., 2004), these data suggest that cells expressing the P2X₇R maintain a higher ATP concentration. Reduction of basal luminescence by oATP pretreatment (5800±1300 cps, n=8) also supports this interpretation. Finally, the effect of BZATP on the human ACN neuroblastoma, a cell line expressing the native P2X₇R, was assayed (Raffaghello L., Pistoia V., Di Virgilio F., unpublished observations). As shown in FIG. 3E, also in this case BZATP induces a large ATP release which is fully blocked by oATP. Basal luminescence total levels in this cell line were 3500±350 cps and 1500±260 cps (n=5), before and after treatment with oATP, respectively, further supporting the finding that cells expressing a functional P2X₇R maintain a higher ATP concentration in the pericellular space.

As an independent proof that HEK293-P2X₇ cells release a larger amount of ATP than mock-transfected HEK293 (HEK293-mock), extracellular ATP was measured in the supernatants using soluble luciferase. Quiescent HEK293-mock cells maintained an extracellular ATP concentration of 80±20 nM (n=12), compared to 220±34 nM (n=10) for HEK-hP2X₇. Addition of BzATP had no effect on the HEK293-mock, but increased extracellular ATP levels to about 400±55 nM (n=10) in the HEK293-hP2X₇ cells supernatants.

Like BzATP, ATP itself should trigger ATP release in the HEK293-P2X₇/pmeLUC cells, since, albeit at high concentrations, ATP is the only known physiological activator of P2X₇ (Di Virgilio et al., 2001; North, 2002). Then, ATP addition to HEK293-P2X₇/pmeLUC cells should trigger an extra increase in luminescence compared to HEK293-pmeLUC cells. The extra increase in luminescence should be due to ATP release via the P2X₇R. FIG. 4 shows that this is the case, whether the transfected receptor is the human or rat P2X₇R. Interestingly, in the HEK293 cells transfected with the rat receptor (FIG. 4A) the extra-increase in luminescence emission (expressed as percent increase over basal) is detectable already at the lowest ATP concentrations used (10-50 μM), while in cells transfected with the human ortholog (FIG. 4B) a luminescence increase over control cells is detectable only at ATP concentrations higher than 100 μM. This is in keeping with the known lower affinity for ATP of the human receptor (Rassendren et al., 1997; Suprenant et al., 1996). If the extra luminescence observed in the HEK293-P2X₇/pmeLUC cells is due to ATP release via P2X₇, then it should be abolished by pre-treatment with oATP. This prediction is fulfilled, as in the oATP-treated cells the luminescence increase matches exactly that of HEK293-pmeLUC (FIGS. 4A and B).

### EXAMPLE 2

### Increase of ATP Release P2X₇R Mediated

One of the most potent stimuli for ATP secretion is plasma membrane stretching. To test ability of pmeLUC to detect stretch-induced ATP release HEK293-P2X₇/pmeLUC and HEK293-pmeLUC cells were exposed to a change in tonicity of the perfusion buffer. Cell monolayers were initially perfused with the usual isotonic solution used in all the experiments, and then switched to a hypotonic buffer, obtained by diluting the standard saline buffer with distilled water (1 to 4) (final tonicity 78 mOsm/L). The tonicity shift causes a clearly detectable release of ATP from both clones (FIG. 5A). However, ATP release is several fold higher in the P2X₇-transfected cells. These cells, as shown in the previous experiments, also maintain a higher basal pericellular ATP level with respect to HEK293-pmeLUC (14200±2300 versus 3150±970 cps, n=7). Interestingly, a peak of ATP release is triggered both by a shift from isotonicity to hypotonicity and from hypotonicity to isotonicity, as well as from a shift from isotonicity to hypertonicity (FIG. 5B). In this case HEK293-hP2X₇/pmeLUC and HEK293-pmeLUC cells were perfused with isotonic buffer followed by a hypertonic solution obtained by dissolving 25 ml of sucrose into 75 ml of standard saline solution (final osmolarity 560 mOsm/L).

These findings suggest that, although membrane stretch-induced ATP release can occur independently of the P2X₇R, it is strongly dependent on the expression of this receptor.

### EXAMPLE 3

### Measurement of ATP Release Induced by Toxic Agents in J774 Macrophage Cells and A549 Pulmonary Epithelial Cells Expressing pmeLUC Probe

### Materials and Methods

Chimeric Probe pmeLUC Construction

Chimeric probe pmeLUC described in Example 1 was used to transfect J774 macrophage cells and A549 pulmonary epithelial cells.

### Cell Line Engineering

In the example shown it was not necessary to previously make the cell line employed expressing any receptor trigger cell response, since this such a cell line constituvely expresses the molecular complex necessary for ATP release. However, it is possible, depending on study necessities, to make the cells expressing a specific receptor for the molecole/drug to be tested to the aim of ensuring an effective binding level, thus simplifying the functional analysis of transduction systems.

### Cellular Transfection

Cells cultured in flasks for cell culture (75 cm²) were transfected with a vector containing the produced chimeric probe, by transfection techniques more suitable for the cell line under examination. As experimental model established macrophage cells, named J774, and established pulmonary epithelial cells, named A459, were used after being transfected with pmeLUC probe using TransFectin (Biorad) that ensure the major percent of positive cells for these cell lines.

### Harvesting of Cells Expressing the Chimeric Probe

36 hours after transfection cells were removed from the bottom of flasks for trypsinization. Cell suspension was transferred in a Falcon tube (from 15 to 50 ml) and centrifuged at 1200 rpm at 20° C. and finally cell precipitate was re-suspended in DMEM medium supplemented with DTT (1 mM).

### Seeding on Multi-Well Plates

50 μl of suspension containing transfected cells (corresponding to about 50.000 cells) were seeded in each well of the plate. Cells were incubated overnight.

### Response Detection

The day after seeding cells were incubated with a saline solution (KRB: Krebs-Ringer modified buffer: 125 mM NaCl, 5 mM KCl, 1 mM Na₃PO₄, 1 mM MgSO₄, 1 mM CaCl₂, 5.5 mM glucose, 20 mM HEPES, pH 7.4, 37° C.) supplemented with luciferin (5 μM) and were directly contacted with a photomultiplier measuring photon emission by luciferase.

### Results

This test was carried out to assay the presence of toxic substances inducing ATP release in live cells.

FIGS. 6 (J774 cells) and 7 (A549 cells) show diagrams wherein it is shown the measurement of ATP release following exposure to several substances.

The example was carried out on two parallel “batch” of cells, J774 and A549:

A) J774 cells/A549 cells expressing pmeLUC probe treated with several ATP concentrations (from 5 μM to 1 mM) (control).

B) J774 cells/A549 cells expressing pmeLUC probe treated with a known toxic agent, bacterial endotoxin (LPS) acting on the constitutive CD14 receptor of both lines (1 μg/ml) (thin dark line).

In absence of outer ATP photon emission has very low intensity. Indeed, in such conditions extracellular ATP concentration, where is localized pmeLUC probe is very low (0.1 μM).

As it is notable from diagrams represented in FIGS. 6A and 7A the addiction of increasing outer ATP concentrations induces a photon emission by the pmeLUC probe proportional to ATP concentration applied to demonstrate the sensitivity of such biosensors for the detection of extracellular ATP.

FIGS. 6B and 7B show ATP release, by J774 cells and A549 cells in response to a toxic agent such as LPS.

It is evident from the diagram that the treatment with toxic substances activated mechanisms capable to induce ATP release at the pericellular level inside cells under examination that is detected by pmeLUC probe.

Such a result shows applicability of such system as cellular biosensor suitable to detect and identify substances causing ATP release such as environmental toxic substances such as LPS and ozone.

### BIBLIOGRAPHY

Adinolfi, E., Kim, M., Young, M. T., Di Virgilio, F., and Surprenant, A. (2003). J. Biol. Chem. 278, 37344-37351.
Anderson, C. M., Bergher, J. P., and Swanson, R. A. (2004). Journal of Neurochemistry 88, 246-256.
Baricordi, O. R., Melchiorri, L., Adinolfi, E., Falzoni, S., Chiozzi, P., Buell, G., and Di Virgilio, F. (1999). J. Biol. Chem. 274, 33206-33208.
Beigi, R., Kobatake, E., Aizawa, M., and Dubyak, G. R. (1999). Am J Physiol Cell Physiol 276, C267-C278.
Brini, M., Pinton, P., King, M. P., Davidson, M., Schon, E. A., and Rizzuto, R. (1999). Nat. Med. 5, 951-954.
Burnstock, G. (2004). Curr. Top. Med. Chem. 4, 793-803.
Di Virgilio, F., Chiozzi, P., Ferrari, D., Falzoni, S., Sanz, J. M., Morelli, A., Torboli, M., Bolognesi, G., and Baricordi, O. R. (2001). Blood 97, 587-600.
Di Virgilio, F., Baricordi, O. R., Romagnoli, R., and Baraldi, P. G. (2004). Curr Drug Targets, 5, 85-99.
Duan, S., Anderson, C. M., Keung, E. C., Chen, Y., Chen, Y., and Swanson, R. A. (2003). 23, 1320-1328.
Ferrari, D., Chiozzi, P., Falzoni, S., Hanau, S., and Di Virgilio, F. (1997). J. Exp. Med. 185, 579-582.
Freyer, D. R., Boxer, L. A., Axtell, R. A., and Todd, R. F. III. (1988). J. Immunol. 141, 580-586.
Hayashi, S., Hazama, A., Dutta, A. K., Sabirov, R. Z., and Okada, Y. (2004). Sci. STKE. 258, 114.
Illes, P., and Norenberg, W. (1993). Trends Pharmacol. Sci. 14, 50-54.
Jouaville, L. S., Pinton, P., Bastianutto, C., Rutter, G. A., and Rizzuto, R. (1999). Proc. Natl. Acad. Sci. USA 96, 13807-13812.
La Sala, A., Ferrari, D., Di Virgilio, F., Idzko, M., Norgauer, J., and Girolomoni, G. (2003). J. Leukoc. Biol. 73, 339-343.
Matzinger, P. (2002). Science 296, 301-305.
Mezghrani, A., Fassio, A., Benham, A., Simmen, T., Braakman, I., and Sitia, R. (2001). EMBO J. 20, 6288-6296.
Murgia, M., Hanau, S., Pizzo, P., Rippa, M., and Di Virgilio, F. (1993). J. Biol. Chem. 268, 8199-8203.
Neary, J. T., Kang, Y., Willoughby, K. A., and Ellis, E. F. (2003). J. Neurosci. 23, 2348-2356.
Newman, E. A. (2001). J. Neurosci. 21, 2215-2223.
North, R. A. (2002). Physiol. Rev. 82, 1013-1067.
Oshimi, Y., Miyazaki, S., and Oda, S. (1999). Immunology 98, 220-227.
Perregaux, D., and Gabel, C. A. (1994). J. Biol. Chem. 269, 15195-15203.
Rassendren, F., Buell, G. N., Virginio, C., Collo, G., North, R. A., and Surprenant, A. (1997). J. Biol. Chem. 272, 5482-5486.
Skoberne, M., Beignon, A. S., and Bhardwaj, N. (2004). Trends. Mol. Med. 10, 251-257.
Solini, A., Chiozzi, P., Morelli, A., Adinolfi, E., Rizzo, R., Baricordi, O. R., and Di Virgilio, F. (2004). Arterioscler. Thromb. Vasc. Biol. 24, 1240-1245.
Surprenant, A., Rassendren, F., Kawashima, E., North, R. A., and Buell, G. (1996). Science 272, 735-738.
Wang, X., Arcuino, G., Takano, T., Lin, J., Peng, W. G., Wan, P., Li, P., Xu, Q., Liu, Q. S., Goldman, S. A., and Nedergaard, M. (2004). Nat. Med. 10, 821-827.
Zanovello, P., Bronte, V., Rosato, A., Pizzo, P., and Di Virgilio, F. (1990). J. Immunol. 145, 1545-1550.
Zimmermann, H. (2000). Naunyn Schmiedebergs Arch. Pharmacol. 362, 299-309.

1. Luminescent chimeric proteins comprising a first N-terminal protein sequence, a second protein sequence and a third C-terminal protein sequence wherein:
(i) said first and said third protein sequence are a leader sequence and an anchor sequence belonging to at least a receptor localized on a plasma membrane site;
(ii) said second protein sequence encodes for the full-length or partial sequence of a photoprotein and is inserted in frame between said first and said third sequence (i); said chimeric protein being addressed to said plasma membrane site of the cell wherein it is expressed.

2. Proteins according to claim 1, wherein said receptor localized on the plasma membrane site is selected from the group that consists in ionic-channel receptors, connexins, G protein coupled receptors, tyrosinekinase activity receptors.

3. Proteins according to claim 1, wherein said photoprotein is selected from the group that consists in luciferase, aequorin, obelin.

4. Proteins according to claim 1, wherein said first and said third protein sequence (i) are the leader sequence and the GPI anchor sequence of the folate receptor and said photoprotein (ii) is luciferase.

5. Proteins according to claim 4, wherein luciferase is fire-fly luciferase.

6. Protein according to claim 5 having aminoacidic sequence of SEQ ID No.1.

7. Nucleotidic sequence encoding for one of the protein according to claim 1.

8. Nucleotidic sequence according to claim 7, having nucleotidic sequence of SEQ ID No.2.

9. Expression vector comprising the nucleotidic sequence according to claim 7.

10. Vector according to claim 9, wherein said vector is selected between pcDNA3, VR 1012.

11. Primary cell culture transfected with the expression vector according to claim 9.

12. Cell line transfected with the expression vector according to claim 9.

13. Cell line according to claim 12, characterized by the native or recombinant expression of a receptor of interest whose activation trigger an increase of extracellular ATP levels.

14. Cell line according to claim 13, wherein said receptor of interest is selected between CD14, P2Y, P2X.

15. Cell line according to claim 14, wherein said receptor P2X is P2X₇.

16. Cell line according to claim 12, selected from the group that consists in HEK 293, HeLa, ACN, N9, N13, PC12, J774, A549.

17. Use of luminescent chimeric proteins according to claim 1, as probes for the measurement of extracellular ATP levels in in vitro systems.

18. Use of luminescent chimeric proteins according to claim 1, as probes for the measurement of extracellular ATP levels in in vivo systems.

19. Use of cell lines according to claim 12 for the measurement of extracellular ATP levels in in vitro systems.

20. Use of cell lines according to claim 12 as experimental model in screening method and/or in analysis method of compounds of interest able to modulate extracellular ATP levels.

21. Screening method of compounds of interest able to modulate extracellular ATP levels comprising the following steps:
a) contacting the cell line as defined according to claim 12, with said compound of interest in the presence of O₂, Mg²⁺ and of the luciferin substrate;
b) detecting cps or luminescence percent over basal value or the value of a preceding stimulation with an agonist as a control and determining the activity as an agonist or antagonist in relation to cps increase or reduction over basal value or the value of a preceding stimulation with an agonist as a control, respectively.

22. Method for the analysis of the presence of toxic substances and/or environmental contaminants able to induce the increase of extracellular ATP levels comprising the following steps:
a) contacting at least a cell line sensible to the toxic substance(s) to be assayed as defined according to claim 12, with a sample to be tested in the presence of O₂, Mg²⁺ and of the luciferin substrate;
b) detecting the presence or the absence of a toxic substance for cells in relation to the cps or luminescence percent increase or reduction over the basal value.

23. Method for the analysis according to claim 22, wherein the cell line of step a) is selected between pulmonary epithelial cells and macrophage cells of mammalian origin, preferably of human origin.

24. Method for the analysis according to claim 23, wherein said human pulmonary epithelial cells are A459 cells.

25. Method for the analysis according to claim 23, wherein said human macrophage cells are J774 cells.

26. Method for the analysis according to claim 22 wherein said toxic substance to be tested is LPS and/or ozone.

27. Biosensor comprising a cell line characterized in that it is transfected with an expression vector according to claim 9.

28. Biosensor comprising a cell line characterized in that it is transfected with an expression vector according to claim 13.

29. Biosensor according to claim 27, wherein said cell line is selected from the group consisting in HEK 293, HeLa, ACN, N9, N13, PC12, J774, A549.

30. Use of biosensor according to claim 27, for the measurement of extracellular levels of ATP in in vitro systems.

31. Biosensor comprising at least one of the luminescent chimeric protein as defined according to claim 1.

32. Use of biosensor according to claim 31, for the measurement of extracellular levels of ATP in in vitro systems.

33. Biosensor comprising a cell line characterized in that it is transfected with an expression vector according to claim 14.

34. Biosensor comprising a cell line characterized in that it is transfected with an expression vector according to claim 15.
